(19) United States
(12) Patent Application Publication
(10) Pub. No.: US 2013/0280310 A1
(43) Pub. Date: Oct. 24, 2013

(54) SHEET-LIKE COSMETIC

(71) Applicants: Kazunobu SUZUKI, Kanagawa (JP); Satoko KISHIMOTO, Kanagawa (JP); Kazuhiko FUJIWARA, Kanagawa (JP); Taira OMORI, Shiga (JP); Masanobu TAKEDA, Shiga (JP)

(72) Inventors: Kazunobu SUZUKI, Kanagawa (JP); Satoko KISHIMOTO, Kanagawa (JP); Kazuhiko FUJIWARA, Kanagawa (JP); Taira OMORI, Shiga (JP); Masanobu TAKEDA, Shiga (JP)

(73) Assignees: TORAY INDUSTRIES, INC.; SHISEIDO COMPANY, LTD.

(21) Appl. No.: 13/453,420

(22) Filed: Apr. 23, 2012

(51) Int. Cl. A61K 8/02; A61Q 19/00

(52) U.S. Cl. 424/401

(57) ABSTRACT

A sheet-like cosmetic is formed by impregnating a laminated sheet having two or more non-woven fabric layer composed of ultra-fine fibers with a cosmetic containing 1 wt % or more of a specific liquid oil component. An average diameter is specified, and select portions are impregnated with a cosmetic containing 1-30 wt % of one, two or more liquid oil components selected from hydrocarbon oils, higher alcohols, higher fatty acids, ester oils, liquid fats/oils, and silicone oils.

### CROSS REFERENCE TO RELATED APPLICATION (S)

None

### DRAWING SELECTED FOR PUBLICATION

None

### BACKGROUND OF THE INVENTION

1. Field of the Invention

The present invention relates to a sheet-like cosmetic. More specifically, it relates to a sheet-like cosmetic that is a laminated sheet having a non-woven fabric layer composed of ultra-fine fibers wherein the sheet is impregnated with a specific cosmetic and thus exhibits superior skin effects such as a skin tightening sensation, plump skin sensation, and a lift-up effect.

2. The Related Art

Known sheet-like cosmetics include face mask sheets that are non-woven fabrics impregnated with cosmetics, wipe sheets, and cleansing sheets. Also known are gel sheets containing polyacrylic and/or a salt thereof and/or bio-cellulose.

In the sheet-like cosmetic field, it is a technology to consider the sensation during use such as softness and texture and usability such as ease of wiping off, adhesion, and ease of pasting on by preparing the non-woven fabric and/or sheets using natural or synthetic ultra-fine fibers or ultra-fine fibers produced by microorganisms.

Unfortunately, conventional sheet-like cosmetics had a technical problem in that not enough effect was felt in terms of a skin tightening sensation, plump skin sensation, and the lift-up effect.

Patent Documents 1 and 2 disclose non-woven fabric for cosmetics such as cleansing sheets, wipe sheets, and face masks containing ultra-fine fibers of 0.1-0.8 dtex.

However, although Patent Documents 1 and 2 describe functions such as softness, texture, and ease of wiping off, they do not investigate improvements in the skin effect at all.

Patent Document 3 discloses wet tissues wherein the wiping-off surface is composed of a material containing split fibrilized ultra-fine fibers and describes improvements in the ease of wiping-off due to the non-woven fabric of the wiping-off surface being composed of the ultra-fine fiber.

However, no consideration is given to the application to sheet-like cosmetics to improve the skin effect.

Patent Document 4 discloses skin cleaning sheets using a three-layer non-woven fabric containing finely split ultra-fine fibers and describes that such sheets have good retention of liquid cleaners, can selectively remove excessive sebum and/or makeup stains, and give a superior sensation during use.

However, no consideration is given to improvements in the skin effect.

Patent Document 5 discloses non-woven sheets having cellulose nano-fibers as the main fibers and describes that the texture is soft.

However, no consideration is given to improvements in the skin effect.

Patent Document 6 discloses sheets for cosmetics composed of polymer ‘nano-fibers’ and describes that they help cosmetic ingredients maintain their activity and are superior in adhesion and the sensation when worn.

However, no consideration is given to improvements in the skin effect.

As described thus far, a sheet-like cosmetic prepared by impregnating a laminated sheet having a non-woven fabric layer of ultra-fine fibers with a cosmetic is limited in the conventional art.

However, a sheet-like cosmetic that solves issues concerning improvements in the skin effects such as a skin tightening sensation, plump skin sensation, and the lift-up effect has not been disclosed in conventional technology.

### RELATED ART DOCUMENTS

Patent Document 1: JP 2008-95223 A
Patent Document 2: JP 2010-195735 A
Patent Document 3: Japanese Patent No. 4575638
Patent Document 4: JP 2002-238797 A
Patent Document 5: JP 2009-185417 A
Patent Document 6: JP 2008-179629 A

### ASPECTS AND SUMMARY OF THE PRESENT INVENTION

### Problem that the Present Invention Aims to Solve

In view of the situation described above, the inventors conducted earnest research on sheet-like cosmetics prepared by impregnating a laminated sheet having a non-woven fabric layer of ultra-fine fibers with a cosmetic and discovered that sheet-like cosmetic prepared by impregnating a sheet having a laminated structure of two or more layers, having a non-woven fabric layer composed of ultra-fine fibers of an average diameter of 1-500 nm at the skin contact surface and additionally having a non-woven fabric layer composed of fibers having an average diameter larger than that of the ultra-fine fibers used in the non-woven fabric layer at the skin contact surface with a cosmetic containing 1 wt % or more of one or more liquid oil components selected from hydrocarbon oils, higher alcohols, higher fatty acids, ester oils, liquid fats/oils, and silicone oils exhibits superior skin effects such as a skin tightening sensation, plump skin sensation, and the lift-up effect and thus completed the present invention.

One aspect of the present invention is to provide a sheet-like cosmetic that has a markedly improved skin effect by impregnating a laminated sheet having a non-woven fabric layer composed of ultra-fine fibers having a specific average diameter at the skin contact surface with a cosmetic containing 1 wt % or more of a specific liquid oil component.

### Means to Solve the Problem

The present invention provides a sheet-like cosmetic prepared by impregnating a sheet that:

(a) is a laminated sheet composed of two or more non-woven fabric layers,

(b) has a non-woven fabric layer composed of ultra-fine fibers having an average diameter of 1-500 nm at the skin contact surface, and

(c) additionally has a non-woven fabric layer composed of fibers having an average diameter larger than that of the ultra-fine fibers used in the non-woven fabric layer at the skin contact surface having an average diameter of 1-500 nm

with a cosmetic containing 1-30 wt % of one, two or more liquid oil components selected from hydrocarbon oils, higher alcohols, higher fatty acids, ester oils, liquid fats/oils, and silicone oils.

Furthermore, the present invention provides the aforementioned sheet-like cosmetic wherein the average diameter of the fibers used in said (c) non-woven fabric layer is 1-50 micrometers and optionally more.

Furthermore, the present invention provides the aforementioned sheet-like cosmetic wherein the impregnation amount of said cosmetic to be impregnated into the non-woven fabric is 5-15 times, and optionally more or less, the mass of the laminated sheet of non-woven fabric.

Also, the present invention provides the aforementioned sheet-like cosmetic wherein said sheet has cuts between the part covering the cheeks and the part covering the face line and is a face mask used by being pasted as the part covering the face line is pulled from the jaw area toward the temple area.

### Effects of the Invention

The sheet-like cosmetic of the present invention can provide a new useful sheet-like cosmetic that is superior in terms of skin effects such as a skin tightening sensation, plump skin sensation, and the lift-up effect by having the aforementioned configuration.

Finally, it is also proposed that the present invention involves a method of manufacturing a sheet-like cosmetic as a sheet-like cosmetic system, wherein the system involves the steps of preparing the noted layers and the step of impregnating and then providing cuts as discussed herein. It is also contemplated that the proposed method includes a step of forming the sheet-like cosmetic system into a face mask form.

### DESCRIPTION OF THE DRAWINGS

None

### DETAILED DESCRIPTION OF THE INVENTION

The sheet-like cosmetic of the present invention is described in detail below. The sheet-like cosmetic of the present invention is used as a face mask for grooming the whole face, a partial sheet mask for grooming the eye area, mouth area, cheeks, and the neck, a wipe-off sheet to remove skin stains, and a cleansing sheet to remove makeup stains.

The sheet used in the present invention is composed of a laminated structure of two or more layers, preferably three layers, including the following (b) and (c).

(b) Having a non-woven fabric layer composed of ultra-fine fibers having an average diameter of 1-500 nm at the skin contact surface, and
(c) additionally having a non-woven fabric layer composed of fibers having an average diameter larger than that of the ultra-fine fibers used in the non-woven fabric layer at the skin contact surface having an average diameter of 1-500 nm.

A preferable three-layer sheet is a sheet that additionally has (d) a sheet having a non-woven fabric layer composed of ultra-fine fibers having an average diameter of 1-500 nm; which three-layer sheet has a laminated structure of (b)/(c)/(d) from the skin contact surface.

Said (b) layer, which is the non-woven fabric layer at the skin contact surface, has a non-woven fabric layer composed of ultra-fine fibers having an average diameter of 1-500 nm. The average diameter of the fibers is preferably 1-200 nm, and more preferably 50-200 nm. By having the non-woven fabric layer composed of the fibers defined by this range at the skin contact surface, a face mask with a high degree of adhesion and a cleansing sheet with a high wipe-off ability can be obtained.

The sheet used in the present invention has the (b) layer, which is the non-woven fabric layer at the skin contact surface, and the (c) layer, a non-woven fabric layer composed of fibers having an average diameter larger than that of the ultra-fine fibers used in the (b) layer. The average diameter of the fibers in the (c) layer is not limited in particular as long as it is bigger than 500 nm; a preferable range is 1 micrometer or more and 50 micrometers or less. By selecting an appropriate fiber diameter, a sheet can be obtained that is suitable for the intended use of the sheet-like cosmetic in terms of handling such as softness, firmness, etc.

The density of the sheet is not necessarily limited; it is preferably 40-150 g/m², and more preferably 60-100 g/m². By selecting an appropriate density, a sheet can be obtained that is superior in terms of ease of handling such as ease of stretching and pasting for a face mask and wear resistance of a wipe-off sheet, thus suitable for the intended use of the sheet-like cosmetic.

Selection of the material of the non-woven fabric that constitutes the sheet is not limited in particular; examples include resin fibers such as polyethylene, polypropylene, polyethylene terephthalate, and nylon, and cellulose fibers such as rayon, cotton, and pulp. The non-woven fabric may be composed of a mixture of two or more kinds of fibers. Also, each of the two or more layers of non-woven fabric may be made of a different fiber from each other.

The liquid oil component contained in the cosmetic to be impregnated into the sheet-like cosmetic is one, two or more liquid oil components selected from hydrocarbon oils, higher alcohols, higher fatty acids, ester oils, liquid fats/oils, and silicone oils. There is no particular limitation about concurrently adding other solid oil components or semi-solid oil components or liquid oil components other than those mentioned above.

Examples of the hydrocarbon oils include liquid paraffin, squalane, olefin oligomer, isodecane, and isododecane.

Examples of the higher alcohols include isostearyl alcohol, 2-octyl dodecanol, 2-decyltetradecanol, hexyldecanol, and oleyl alcohol.

Examples of the higher fatty acids include isostearic acid, linolic acid, linoleic acid, and arachidonic acid.

Examples of the ester oils include isopropyl myristate, cetyl octanoate, octyldodecyl myristate, isopropyl palmitate, and cetyl 2-ethylhexanoate.

Examples of the liquid fats and oils include avocado oil, tsubaki oil, macadamia nut oil, corn oil, olive oil, safflower oil, cotton seed oil, soybean oil, tea seed oil, jojoba oil, germ oil, and meadow foam oil.

Examples of the silicone oils include dimethylpolysiloxane, methylphenylpolysiloxane, decamethylpolysiloxane, dodecamethylpolysiloxane, amino-modified polysiloxane, polyether-modified polysiloxane, and fluorine-modified polysiloxane.

Particularly preferable liquid oil components include liquid paraffin, squalane, olefin oligomer, isostearyl alcohol, 2-octyldodecanol, isostearic acid, cetyl 2-ethylhexanoate, meadow foam oil, dimethylpolysiloxane, methylphenylpolysiloxane, and amino-modified polysiloxane.

The blend ratio of the liquid oil component contained in the cosmetic to be impregnated into the sheet is not limited in particular as long as it is 1 wt % or more relative to the total amount of the cosmetic; the upper limit is 30 wt %. If it is less than 1 wt %, then a sufficient skin effect such as the plump skin sensation and lift-up effect is difficult to obtain. It is preferably 1-30 wt %, more preferably 3-15 wt %.

In addition to the aforementioned oil components, other ingredients used in cosmetics can be blended as necessary in the cosmetic that impregnates the sheet; examples include whitening agents, rough skin care agents, anti-wrinkling agents, amino acids, plant extracts, ultraviolet absorbents, surfactants, humectants, thickeners, alcohols, powders, coloring agents, pH adjusting agents, stabilizers, preservatives, perfumes, and water.

The cosmetic to be impregnated into the sheet is preferably a liquid cosmetic, but the formulation form is not limited in particular as long as it is a cosmetic that can be used for impregnation. For example, cosmetics such as lotions, emulsions, essences, and creams are preferably used for impregnation.

The impregnation amount of the cosmetic to be impregnated into the sheet is not limited in particular; it is adjusted appropriately according to the characteristics of the sheet and the cosmetic; the amount of impregnation of the cosmetic liquid to be impregnated into the sheet of the said sheet-like cosmetic is preferably 5-15 times of the mass of the sheet having the non-woven fabric laminated structure.

The sheet-like cosmetic of the present invention is preferably used as a face mask for grooming the whole face or a partial sheet mask for grooming the eye area, mouth area, cheeks, and the neck.

The present invention most preferably is a face mask wherein the sheet has cuts between the part covering the cheeks and the part covering the face line and is a face mask used by being pasted as the part covering the face line is pulled from the jaw area toward the temple area.

### EXAMPLES

The invention is described in detail through Examples below, but the invention shall not be limited to them. The blend ratios in Examples are in wt % units unless specified otherwise.

Essences having compositions shown in Examples 1-3 and Comparative examples 1-3 in Table 1 were prepared; face masks were prepared by impregnating sheets with these essences, which were then pasted on faces of a panel of ten women; and the skin effects (adhesion and also skin tightening sensation, plump skin sensation, the lift-up effect, and absence of stickiness after the mask was peeled off 10 minutes after pasting) were evaluated. The results are shown in Table 1.

Also, the same evaluation was conducted for the sheets impregnated with essences of Example 2 in Table 1 and the results are shown in Table 2.

The sheets used in Table 1 have a three-layer non-woven fabric structure composed of, from the contact surface to the face, nylon ultra-fine fibers having an average diameter of 1-500 nm/nylon fibers having an average diameter of 1-50 micrometers/nylon ultra-fine fibers having an average diameter of 1-500 nm.

Also, the sheets used in Table 2 are one layer non-woven fabric sheets composed of cotton having an average diameter of 20 micrometers for Comparative example 4, and one layer non-woven fabric sheets composed of mixed rayon, cotton, and PET having an average diameter of 20 micrometers for Comparative example 5.

In terms of the sheet shape, the face mask that is used is a face mask wherein the sheet has cuts between the part covering the cheeks and the part covering the face line and the face mask is pasted as the part covering the face line is pulled from the jaw area toward the temple area.

For each test item to demonstrate the effects of the present invention, a panel of ten women specialists made an evaluation based on the following evaluation point criteria, and a four step evaluation scale was established using the total of the evaluation points. The results are also shown in Table 1.

### [Evaluation Point Criteria]

5 points: Exceptionally superior
4 points: Excellent
3 points: Midway
2 point: Inferior
1 point: Very inferior

### [Evaluation Criteria]

A: The total score is 40 points or higher.
B: The total score is 30-39 points.
C: The total score is 20-39 points.
D: The total score is 19 points or lower.

TABLE 1

| | | | | Comparative | Comparative | Comparative |
|---|---|---|---|---|---|---|
| Raw material name | Example 1 | Example 2 | Example 3 | example 1 | example 2 | example 3 |
| Liquid petrolatum | 0.5 | 1.0 | — | — | 0.4 | — |
| Squalane | 0.5 | 1.0 | 5.0 | — | 0.4 | — |
| Isostearic acid | — | — | 4.0 | — | — | — |
| Cetyl 2-ethylhexanoate | — | — | 1.0 | — | — | — |
| Meadow foam oil | — | — | 3.0 | — | — | — |
| Dimethylsiloxane | — | 1.0 | 2.0 | — | — | — |
| Petrolatum | — | — | — | — | — | 3.0 |
| Glycerin | 5.0 | 5.0 | 5.0 | 5.0 | 5.0 | 5.0 |
| Butylene glycol | 10.0 | 10.0 | 10.0 | 10.0 | 10.0 | 10.0 |
| Polyethylene glycol 4000 | 3.0 | 3.0 | 3.0 | 3.0 | 3.0 | 3.0 |
| Hyaluronic acid | 0.01 | 0.01 | 0.01 | 0.01 | 0.01 | 0.01 |
| PEG-60 Hydrogenated castor oil | 0.5 | 0.5 | 0.5 | 0.5 | 0.5 | 0.5 |
| Carboxyvinyl polymer | 0.2 | 0.2 | 0.2 | 0.2 | 0.2 | 0.2 |
| Xanthan gum | 0.2 | 0.2 | 0.2 | 0.2 | 0.2 | 0.2 |
| Sodium hydroxide | 0.05 | 0.05 | 0.05 | 0.05 | 0.05 | 0.05 |
| Yeast extract | 0.1 | 0.1 | 0.1 | 0.1 | 0.1 | 0.1 |
| Zingiber Cassumunar extract | 0.1 | 0.1 | 0.1 | 0.1 | 0.1 | 0.1 |
| Edetate | 0.1 | 0.1 | 0.1 | 0.1 | 0.1 | 0.1 |
| Methylparaben | 0.2 | 0.2 | 0.2 | 0.2 | 0.2 | 0.2 |
| Ion-exchanged water | Balance | Balance | Balance | Balance | Balance | Balance |
| Impregnation amount of the | 10 | 10 | 10 | 10 | 10 | 10 |
| cosmetic/sheet weight | | | | | | |
| Adhesion | A | A | A | A | A | A |
| Skin tightening sensation | A | A | A | A | A | B |
| Plump skin sensation | B | A | A | D | C | A |
| Lift-up effect | B | A | A | D | C | B |
| Absence of the sticky sensation | A | A | B | A | A | D |

For the sheets, three-layer sheets of nylon ultra-fine fibers/nylon fibers/nylon ultra-fine fibers (density 70 g/m²) were used.

TABLE 2

| | Comparative | Comparative |
|---|---|---|
| | example 4 | example 5 |
| Sheet | Cotton | Rayon/ |
| | (Density 70 | Cotton/PET |
| | g/m²) | (Density 70 |
| | | g/m²) |
| Impregnation amount of the | 10 | 10 |
| cosmetic/sheet weight | | |
| Adhesion | C | C |
| Skin tightening sensation | C | D |
| Plump skin sensation | B | B |
| Lift-up effect | B | B |
| Absence of the sticky | B | B |
| sensation | | |

The essence that was impregnated into the sheet was the essence of Example 2 in Table 1.

For the sheet, Comparative example 4 used a one layer non-woven fabric sheet composed of cotton having an average diameter of 20 micrometers. Comparative example 5 used a one layer non-woven fabric sheet composed of mixed fibers of rayon, cotton, and PET having an average diameter of 20 micrometers.

The results shown in Table 1 and Table 2 indicate that a sheet-like cosmetic that has markedly improved skin effects (adhesion and also skin tightening sensation, plump skin sensation, and the lift-up effect after the mask was peeled off 10 minutes after pasting) can be provided by impregnating a laminated sheet having a non-woven fabric layer composed of ultra-fine fibers having a specific average diameter with a cosmetic containing 1 wt % or more of a specific liquid oil component.

### INDUSTRIAL APPLICATIONS

The sheet-like cosmetic of the present invention is a new useful sheet-like cosmetic that has markedly improved skin effects by impregnating a laminated sheet having a non-woven fabric layer composed of ultra-fine fibers having a specific average diameter at the skin contact surface with a cosmetic containing 1 wt % or more of a specific liquid oil component. In particular, it is most preferably used as a face mask having a specific shape.

What is claimed is:

1. A sheet-like cosmetic, comprising:
a sheet that (a) is a laminated sheet composed of two or more non-woven fabric layers, (b) has a non-woven fabric layer composed of ultra-fine fibers having an average diameter of 1-500 nm at a skin contact surface, and (c) additionally has a non-woven fabric layer composed of fibers having an average diameter larger than that of the ultra-fine fibers used in the non-woven fabric layer at the skin contact surface having an average diameter of 1-500 nm wherein which sheet is impregnated with a cosmetic containing 1-30 wt % of one, two or more liquid oil components selected from hydrocarbon oils, higher alcohols, higher fatty acids, ester oils, liquid fats/oils, and silicone oils.

2. The sheet-like cosmetic, according to claim 1, wherein:
the average diameter of the fibers used in said (c) non-woven fabric layer is 1-50 micrometers.

3. The sheet-like cosmetic, according to claim 1, wherein:
the impregnation amount of said cosmetic to be impregnated into the non-woven fabric is 5-15 times the mass of the laminated sheet of non-woven fabric.

4. The sheet-like cosmetic, according to claim 2, wherein:
the impregnation amount of said cosmetic to be impregnated into the non-woven fabric is 5-15 times the mass of the laminated sheet of non-woven fabric.

5. The sheet-like cosmetic, according to claim 1, wherein:
said sheet has cuts between the part covering the cheeks and the part covering the face line and which sheet-like cosmetic is a face mask used by being pasted as the part covering the face line is pulled from the jaw area toward the temple area.

6. The sheet-like cosmetic, according to claim 2, wherein:
said sheet has cuts between the part covering the cheeks and the part covering the face line and which sheet-like cosmetic is a face mask used by being pasted as the part covering the face line is pulled from the jaw area toward the temple area.

7. The sheet-like cosmetic, according to claim 3, wherein:
said sheet has cuts between the part covering the cheeks and the part covering the face line and which sheet-like cosmetic is a face mask used by being pasted as the part covering the face line is pulled from the jaw area toward the temple area.

8. The sheet-like cosmetic, according to claim 4, wherein:
said sheet has cuts between the part covering the cheeks and the part covering the face line and which sheet-like cosmetic is a face mask used by being pasted as the part covering the face line is pulled from the jaw area toward the temple area.

9. A sheet-like cosmetic system, comprising:
a sheet that further comprises:
(a) a laminated sheet, composed of at least two non-woven fabric layers;
(b) at least one said non-woven fabric layer composed of ultra-fine fibers having a first average diameter of 1-500 nm proximate an operative skin contact surface, and
(c) at least one additional non-woven fabric layer composed of fibers having a second average diameter larger than that of the ultra-fine fibers used in the non-woven fabric layer at the skin contact surface having said first average diameter of 1-500 nm, and
(d) wherein said sheet is impregnated with an impregnation amount of a cosmetic containing 1-30 wt % of one, two or more liquid oil components selected from a group consisting of: hydrocarbon oils, higher alcohols, higher fatty acids, ester oils, liquid fats/oils, and silicone oil;
(e) wherein, an impregnation mass of said impregnation amount of said cosmetic impregnated into said sheet is 5-15 times a mass of said laminated sheet.

10. A sheet-like cosmetic system, according to claim 9, further comprising:
an operative face mask portion formed by said sheet;
a plurality of cuts on said sheet;
said cuts being bounded openings through said sheet proximate a location between a pair of opposing regions covering proximate a user's cheeks and a part covering a face line, whereby said face mask portion is operative during said a use by being pasted as the part covering said face line is pulled from a user's jaw area toward a user's temple area.
